(19) United States
(12) Patent Application Publication
(10) Pub. No.: US 2013/0115189 A1
(43) Pub. Date: May 9, 2013

(54) Use of Interleukin-1 Beta Mutein Conjugates in the Treatment of Diabetes

(71) Applicants: Martin F. Bachmann, Ramismuhle (CH); Gunther Spohn, Zurich (CH); Patrik Maurer, Winterthur (CH)

(72) Inventors: Martin F. Bachmann, Ramismuhle (CH); Gunther Spohn, Zurich (CH); Patrik Maurer, Winterthur (CH)

(73) Assignee: Cytos Biotechnology AG, Zurich-Schlieren (CH)

(21) Appl. No.: 13/395,099

(22) PCT Filed: Sep. 9, 2010
(86) PCT No.: PCT/EP2010/063237

§ 371 Date: Oct. 19, 2012

(30) Foreign Application Priority Data
Sep. 10, 2009 (EP) 09169989.2

(51) Int. Cl. A61K 39/385

(52) U.S. Cl. CPC A61K 39/385
USPC 424/85.2

(57) ABSTRACT

The present invention provides compositions, pharmaceutical compositions and vaccines for the treatment, amelioration and/or prophylaxis of type II diabetes. The compositions, pharmaceutical compositions and vaccines of the invention comprise a virus-like particle of an RNA bacteriophage and an antigen, wherein said antigen comprises an interleukin-1 beta (IL-1β) mutein. When administered to an animal, preferably to a human, said compositions, pharmaceutical compositions, and vaccines induce efficient immune responses, in particular antibody responses, wherein typically and preferably said antibody responses are directed against IL-1β. Thus, the invention provides methods of treating, ameliorating or preventing type II diabetes by way of active immunization against IL-1β.

### FIELD OF THE INVENTION

The present invention provides compositions, pharmaceutical compositions and vaccines for the treatment, amelioration and/or prophylaxis of type II diabetes. The compositions, pharmaceutical compositions and vaccines of the invention comprise a virus-like particle of an RNA bacteriophage and an antigen, wherein said antigen comprises an interleukin-1 beta (IL-β) mutein. When administered to an animal, preferably to a human, said compositions, pharmaceutical compositions, and vaccines induce efficient immune responses, in particular antibody responses, wherein typically and preferably said antibody responses are directed against IL-1β. Thus, the invention provides methods of treating, ameliorating or preventing type II diabetes by way of active immunization against IL-1β.

### RELATED ART

Type 2 diabetes is a chronic metabolic disorder characterized by the presence of hyperglycemia due to defective insulin secretion, insulin action or a combination of both. Although the mechanisms of pancreatic β-cell failure in type 2 diabetes are not fully elucidated, stress and inflammatory pathways have been implicated. Metabolic stress caused by repetitive glucose excursions, dyslipidemia and adipokines can induce an inflammatory response in the pancreas characterized by local cytokine secretion, islet immune-cell infiltration, β-cell apoptosis, amyloid deposits and fibrosis. IL-1β has emerged as a master cytokine, which regulates islet chemokine production and causes impaired insulin production and β-cell death. Blockade of IL-1 signalling by administration of recombinant IL-1 receptor antagonist or neutralizing monoclonal antibodies has been shown to improve glycemic control in animal models of type 2 diabetes (Sauter et al. 2008, Endocrinology, Vol. 149(5) pp. 2208-18; Osborn et al. 2008, Cytokine, Vol. 44(1) pp. 141-8). Furthermore, treatment of type 2 diabetes patients with recombinant human IL-1 receptor antagonist (Anakinra) resulted in a decrease of glycated haemoglobin levels (a reliable readout for long term glycemia) and improved β-cell function (Larsen et al. 2007, N Engl J Med, Vol. 356(15) pp. 1517-1526, 2007).

### SUMMARY OF THE INVENTION

We have found that the inventive compositions comprising at least one IL-1β mutein are not only capable of inducing immune responses against IL-1β, and hereby in particular antibody responses, but are, furthermore, capable of neutralizing the pro-inflammatory activity of IL-1β in vivo. This has been demonstrated in a mouse model (cf. Example 3) as well as in a monkey model (cf. Example 11), which is believed to closely resemble the situation in humans.

We have now surprisingly found that active immunization with a composition of the invention resulted in the amelioration of the diet-induced diabetic phenotype in a mouse model (cf. Surwit et al., DIABETES, Vol. 37, 1988, 1163-1167) of diabetes (cf. Example 5). Furthermore, it was surprisingly found that the receptor binding and, thus, the biological activity of human IL-1β can be reduced by exchanging its N-terminal amino acid residue with the amino acid sequence MDI (Example 6). It was also found that the mutated N-terminus synergistically interacts with the D145K mutation, a mutation which is known to reduce the biological activity of human IL-1β (Example 6). An IL-1β mutein comprising both mutations showed extraordinarily low biological activity. In a primate study, human IL-1β mutein comprising the D145K mutation and the amino acid sequence MDI at the N-terminus showed significantly reduced reactogenicity as compared to wildtype human and primate IL-1β (Example 7). Low biological activity of the IL-1β mutein reduces the reactogenicity of the vaccine in vivo (cf. Example 10) and, thus, ultimately contributes to the safety of the vaccine.

Thus, one aspect of the invention is a composition for use in a method of treating diabetes, preferably type II diabetes, said composition comprising: (a) a virus-like particle of an RNA bacteriophage with at least one first attachment site; and (b) at least one antigen with at least one second attachment site, wherein said at least one antigen comprises or preferably consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue; and wherein (a) and (b) are linked through said at least one first and said at least one second attachment site.

Further aspects of the invention are vaccine compositions, pharmaceutical compositions, methods and uses as disclosed below.

### DETAILED DESCRIPTION OF THE INVENTION

Adjuvant: The term “adjuvant” as used herein refers to non-specific stimulators of the immune response or to substances that allow the generation of a depot in the host which, when combined with the vaccine or with the pharmaceutical composition, respectively, may provide for an even more enhanced immune response. Preferred adjuvants are complete and incomplete Freund's adjuvant, aluminum containing adjuvant, preferably aluminum hydroxide, most preferably alum, and modified muramyldipeptide. Further preferred adjuvants are mineral gels such as aluminum hydroxide, surface active substances such as lysolecithin, pluronic polyols, polyanions, peptides, oil emulsions, keyhole limpet hemocyanins, dinitrophenol, and human adjuvants such as BCG (bacille Calmette Guerin) and Corynebacterium parvum. Further adjuvants that can be administered with the compositions of the invention include, but are not limited to, monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The term adjuvant also encompasses mixtures of these substances. VLPs have been generally described as an adjuvant. However, the term “adjuvant”, as used within the context of this application, refers to an adjuvant not being the VLP used for the inventive compositions, rather it relates to an additional, distinct component. In particular, the term adjuvant shall not refer to a virus-like particle of an RNA bacteriophage.

Antigen: As used herein, the term “antigen” refers to a molecule capable of being bound by an antibody or a T-cell receptor (TCR) if presented by MHC molecules. An antigen is capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. The term “antigen” as used herein preferably refers to a polypeptide wherein said polypeptide comprises or consists of an IL-1β mutein. The term “antigen” as used herein however shall not refer to a virus-like particle, and in particular not to a virus-like of an RNA bacteriophage.

Specific binding (antibody/antigen): Within this application, antibodies are defined to be specifically binding if they bind to the antigen with a binding affinity (Ka) of 10⁶ M⁻¹ or greater, preferably 10⁷ M⁻¹ or greater, more preferably 10⁸ M⁻¹ or greater, and most preferably 10⁹ M⁻¹ or greater. The affinity of an antibody can for example be determined by by Scatchard analysis, by ELISA, or by Biacore analysis.

Specific binding (IL-β/IL-1 receptor): The interaction between a receptor and a receptor ligand can be characterized by biophysical methods generally known in the art, including, for example, ELISA or Biacore analysis. An IL-1β molecule, including an IL-1β mutein, is regarded as capable of specifically binding an IL-1 receptor, when the binding affinity (Ka) of said IL-1β molecule or of said IL-1β mutein to said IL-1 receptor is at least 10⁵ M⁻¹, preferably at least 10⁶ M⁻¹, more preferably at least 10⁷ M⁻¹, still more preferably at least 10⁸ M⁻¹, and most preferably at least 10⁹ M⁻¹; wherein preferably said IL-1 receptor is a human IL-1 receptor. Very preferably, said IL-1 receptor comprises or more preferably consists of any one of the sequences SEQ ID NO:1 or SEQ ID NO:2, wherein further preferably aid IL-1 receptor comprises or more preferably consists of SEQ ID NO:1.

Associated: The terms “associated” or “association” as used herein refer to all possible ways, preferably chemical interactions, by which two molecules are joined together. Preferably, association is by way of covalent interactions, wherein further preferably said covalent interactions are selected from ester bonds, ether bonds, phosphoester bonds, amide bonds, peptide bonds, carbon-phosphorus bonds, carbon-sulfur bonds such as thioether, or imide bonds.

Attachment Site, First: As used herein, the phrase “first attachment site” refers to an element which is naturally occurring with the VLP of an RNA bacteriophage, or which is artificially added to the VLP of an RNA bacteriophage, and to which the second attachment site may be linked. The first attachment site preferably comprises or still more preferably is an amino acid residue or a chemically reactive group such as an amino group. Preferably, the first attachment site is an amino group of an amino acid residue. In a further preferred embodiment the first attachment site is lysine residue. In a still further preferred embodiment the first attachment site is an amino group of a lysine residue. In a preferred embodiment said first attachment site is the amino group of a lysine residue, wherein preferably said lysine residue is a lysine residue which is naturally occurring with said VLP of an RNA bacteriophage. The first attachment site is typically and preferably located on the surface, and further preferably on the outer surface of the VLP of an RNA bacteriophage, most preferably of RNA bacteriophage Qβ. Multiple first attachment sites are present on the surface, preferably on the outer surface of the VLP of an RNA bacteriophage, most preferably of the VLP of RNA bacteriophage Qβ, typically and preferably in a repetitive configuration. In a preferred embodiment the first attachment site is associated with the VLP of an RNA bacteriophage, through at least one covalent bond, preferably through at least one peptide bond. In a further preferred embodiment the first attachment site is naturally occurring with the VLP of an RNA bacteriophage, preferably with the VLP of RNA bacteriophage Qβ. In a preferred embodiment the first attachment site is associated with said VLP of an RNa bacteriophage through at least one covalent bond, preferably through at least one peptide bond, wherein preferably said RNA bacteriophage is Qβ. In a further preferred embodiment said first attachment site is the amino group of a lysine residue, wherein said lysine residue is a lysine residue of a coat protein of an RNA bacteriophage, most preferably of RNA bacteriophage Qβ. In a further preferred embodiment said first attachment site is an amino group of a lysine residue of a coat protein of an RNA bacteriophage, wherein preferably said coat protein comprises or preferably consists of the amino acid sequence of SEQ ID NO:3. In a further preferred embodiment said first attachment site is a lysine residue, wherein said lysine residue is a lysine residue of a coat protein of an RNA bacteriophage, most preferably of RNA bacteriophage Qβ.

Attachment Site, Second: As used herein, the phrase “second attachment site” refers to an element which is naturally occurring with or which is artificially added to the antigen, preferably to the IL-1β mutein comprised by said antigen, and to which the first attachment site may be linked. The second attachment site preferably is an amino acid residue. More preferably, the second attachment site is a chemically reactive group, preferably a chemically reactive group of an amino acid residue. Very preferably, said second attachment site is a sulfhydryl group, preferably a sulfhydryl group of a cysteine residue. The term “antigen with at least one second attachment site” refers, therefore, to a construct comprising an antigen, preferably an IL-1β mutein and at least one second attachment site. However, in particular for a second attachment site which is not naturally occurring within the antigen, such a construct typically and preferably further comprises a “linker”. In preferred embodiment the second attachment site is associated with the antigen, preferably with the IL-1β mutein, through at least one covalent bond, preferably through at least one peptide bond. In a further embodiment, the second attachment site is naturally occurring within the antigen, preferably within the IL-1β mutein. In another further preferred embodiment, the second attachment site is artificially added to the IL-1β mutein, preferably through a linker, wherein further preferably said linker comprises or alternatively consists of a cysteine residue. Very preferably said linker is fused to the IL-1β mutein by way of a peptide bond.

Linked: The terms “linked” or “linkage” as used herein, refer to all possible ways, preferably chemical interactions, by which the at least one first attachment site and the at least one second attachment site are joined together. Linkage preferably is by way of covalent interactions. Covalent interactions preferably are covalent bonds selected from ester bonds, ether bonds, phosphoester bonds, amide bonds, peptide bonds, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide bond(s). The term “linked” as used herein, however, shall not only refer to a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker Thus, in a preferred embodiment said least one first attachment site and said at least one second attachment site are covalently linked via least one, preferably exactly one, heterobifunctional cross-linker, wherein preferably said first attachment site is the amino group of a lysine residue, and wherein further preferably said second attachment site is the sulfhydryl group of a cysteine residue.

Linker: A “linker”, as used herein, either associates the second attachment site with the IL-1β mutein or comprises or consists of the second attachment site. Preferably, a “linker”, as used herein, comprises the second attachment site, typically and preferably—but not necessarily—as one amino acid residue, preferably as a cysteine residue. In a preferred embodiment said linker is an amino acid linker. In a preferred embodiment said linker comprises exactly one cysteine residue and said second attachment site is the sulfhydryl group of said exactly one cysteine residue. Association of the linker with the IL-1β mutein is preferably by way of at least one covalent bond, more preferably by way of at least one peptide bond.

Amino acid linker: The term “amino acid linker” refers to a linker comprising least one amino acid residue. In a preferred embodiment said amino acid linker consists exclusively of amino acid residues.

Ordered and repetitive antigen array: As used herein, the term “ordered and repetitive antigen array” generally refers to a repeating pattern of antigen or to a structure which is characterized by a typically and preferably high order of uniformity in spatial arrangement of the antigens with respect to virus-like particle, respectively. In one embodiment of the invention, the repeating pattern may be a geometric pattern. Certain embodiments of the invention, such as antigens coupled to the VLP of RNA bacteriophages, are typical and preferred examples of suitable ordered and repetitive antigen arrays which, moreover, possess strictly repetitive paracrystalline orders of antigens, preferably with spacings of 1 to 30 nanometers, preferably 2 to 15 nanometers, even more preferably 2 to 10 nanometers, even again more preferably 2 to 8 nanometers, and further more preferably 1.6 to 7 nanometers.

human IL-1β: The term human IL-1β refers to any polypeptide consisting of an amino acid sequence which is at least 90%, preferably at least 95%, more preferably at least 96%, still more preferably at least 97%, still more preferably at least 98%, still more preferably at least 99%, and most preferably 100% identical to human IL-1β 116-269 (SEQ ID NO:4). In a very preferred embodiment human IL-1β refers to a polypeptide consisting of SEQ ID NO:4. Human IL-1β also includes human IL-1β which is recombinantly expressed. Polypeptides which are expressed in a prokaryotic expression system such as E. coli are typically characterized by an N-terminal methionine residue. Thus, the term human IL-1β further preferably refers to SEQ ID NO:20 and to any mixture of SEQ ID NO:20 and SEQ ID NO:4.

IL-1β mutein: The term “IL-1β mutein” as used herein refers to a polypeptide consisting of a mutated amino acid sequence, wherein the amino acid sequence to be mutated is human IL-1β. Typically and preferably an IL-1β mutein comprise a biological activity of less than 80%, more preferably of less than 60%, still more preferably of less than 40%, still more preferably of less than 20%, and most preferably of less than 10% of the biological activity of a polypeptide consisting of said amino acid sequence to be mutated, wherein preferably said biological activity is determined by the capacity of said polypeptides to induce IL-6 formation in human cells, preferably in PBMCs or HeLa cells. When introduced into an animal, the inventive compositions comprising a preferred IL-1β mutein typically and preferably induce antibodies comprising cross reactivity to a polypeptide consisting of said amino acid sequence to be mutated. Thus, when introduced into an animal, inventive compositions comprising a preferred IL-1β mutein typically and preferably induce antibodies capable of specifically binding human IL-1β, preferably SEQ ID NO:4. Preferred in the context of the invention are IL-1β muteins comprising a mutated N-terminus. Very preferably, IL-1β muteins comprise the N-terminal amino acid sequence MDI (SEQ ID NO:5). Very preferably, an IL-1β mutein consists of a mutated amino acid sequence, wherein the N-terminal amino acid residue of said amino acid sequence to be mutated is replaced by the amino acid sequence MDI (SEQ ID NO:5). Further preferably, IL-1β muteins comprise at least on, preferably exactly one further mutation causing a reduced biological activity of the IL-1β mutein as compared to a polypeptide consisting of said amino acid sequence to be mutated. Very preferably an IL-1β mutein consists of a mutated amino acid sequence, wherein the amino acid sequence to be mutate is human IL-1β, preferably SEQ ID NO:4, and wherein the amino acid residue in position 145 of said amino acid sequence to be mutated, preferably of SEQ ID NO:4, is exchanged by another amino acid residue. Still further preferably an IL-1β mutein consists of a mutated amino acid sequence, wherein the amino acid sequence to be mutate is human IL-1β, preferably SEQ ID NO:4, wherein the amino acid residue in position 145 of said amino acid sequence to be mutated, preferably of SEQ ID NO:4, is an aspartic acid residue, and wherein said aspartic acid residue is exchanged by a lysine residue. Still further preferably an IL-1β mutein consists of a mutated amino acid sequence, and wherein the amino acid sequence to be mutate is human IL-1β, preferably SEQ ID NO:4, and wherein the N-terminal amino acid residue of said amino acid sequence to be mutated is replaced by the amino acid sequence MDI (SEQ ID NO:5), and wherein further the amino acid residue in position 145 of said amino acid sequence to be mutated, preferably of SEQ ID NO:4, is exchanged by another amino acid residue, preferably by a lysine residue.

Amino acid exchange: the expression amino acid exchange refers to the exchange of an amino acid residue in a certain position of an amino acid sequence by any other amino acid residue.

biological activity: The terms “biological activity” or “biologically active” as used herein with respect to a IL-1β molecule, including the IL-1β mutein, refer to the ability of the IL-1β molecule and/or of the IL-1β mutein to induce the production of IL-6 after systemical administration into an animal, preferably into a human. Preferably, the capability of an IL-1β molecule and/or of an IL-1β mutein to induce IL-6 formation in vivo is determined as outlined in Examples 2A and 7. The terms “biological activity” or “biologically active” also refer the ability of an IL-1β molecule and/or of an IL-1β mutein to induce the proliferation of thymocytes (Epps et al., Cytokine 9(3):149-156 (1997), D10.G4.1 T helper cells (Orencole and Dinarello, Cytokine 1(1):14-22 (1989), or the ability to induce the production of IL-6 from MG64 or HaCaT cells (Boraschi et al., J. Immunol. 155:4719-4725 (1995) or fibroblasts (Dinarello et al., Current Protocols in Immunology 6.2.1-6-2-7 (2000)), or the production of IL-2 from EL-4 thymoma cells (Simon et al., J. Immunol. Methods 84(1-2):85-94 (1985)), or the ability to inhibit the growth of the human melanoma cell line A375 (Nakai et al., Biochem. Biophys. Res. Commun. 154:1189-1196 (1988)). Very preferably, the term biological activity of an IL-1β molecule or of an IL-1β mutein refers to their capacity to induce IL-6 formation in human cells, preferably in PBMCs or in HeLa cells.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product.

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed.

Coat protein: The term “coat protein” refers to a viral protein, preferably a subunit of a natural capsid of a virus, preferably of an RNA bacteriophage, which is capable of being incorporated into a virus capsid or a VLP. Coat proteins are also known as capsid proteins.

Recombinant VLP: The term “recombinant VLP”, as used herein, refers to a VLP that is obtained by a process which comprises at least one step of recombinant DNA technology.

Virus-like particle (VLP), as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. In one embodiment, a virus-like particle is a virus particle, in which the viral genome has been physically or chemically inactivated. Typically and more preferably a virus-like particle lacks all or part of the replicative and infectious components of the viral genome. A virus-like particle in accordance with the invention may contain nucleic acid which is distinct from the genome. A typical and preferred embodiment of a virus-like particle in accordance with the present invention is a viral capsid such as the viral capsid of the corresponding virus, preferably bacteriophage, most preferably RNA bacteriophage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular. For example, the capsids of RNA bacteriophages have a spherical form of icosahedral symmetry. The term “capsid-like structure” as used herein, refers to a macromolecular assembly composed of viral protein subunits resembling the capsid morphology in the above defined sense but deviating from the typical symmetrical assembly while maintaining a sufficient degree of order and repetitiveness. Thus a typical feature of a virus-like particle is the highly ordered and repetitive arrangement of its subunits.

Virus-like particle of an RNA bacteriophage: As used herein, the term “virus-like particle of an RNA bacteriophage” refers to a virus-like particle comprising, or preferably consisting essentially of, or consisting of coat proteins, mutants or fragments thereof, of an RNA bacteriophage. In addition, a virus-like particle of an RNA bacteriophage is resembling the structure of an RNA bacteriophage. Furthermore, a virus-like particle of an RNA bacteriophage is non replicative and/or non-infectious. Typically and preferably a virus-like particle of an RNA bacteriophage is lacking at least one of the genes, preferably all genes, encoding the replication machinery of the RNA bacteriophage. Further preferably a virus-like particle of an RNA bacteriophage is also lacking the gene or genes encoding the protein or proteins responsible for viral attachment to or entry into the host. This definition, however, also encompasses virus-like particles of RNA bacteriophages, in which the aforementioned gene or genes are still present but inactive, and, therefore, also are leading to non-replicative and/or non-infectious virus-like particles of an RNA bacteriophage. Preferred VLPs derived from RNA bacteriophages exhibit icosahedral symmetry and consist of 180 subunits (monomers). Preferred methods to render a virus-like particle of an RNA bacteriophage non replicative and/or non-infectious is by physical, chemical inactivation, such as UV irradiation, formaldehyde treatment, typically and preferably by genetic manipulation.

diabetes: The term diabetes refers to any type of diabetes mellitus. Preferably diabetes refers to type I diabetes and/or type II diabetes. Most preferably diabetes refers to type II diabetes.

dose: The term dose refers to the total amount of the composition of the invention, of the vaccine composition of the invention, or of the pharmaceutical composition of the invention which is administered to an animal, preferably to a human in one day. Typically and preferably, but not necessarily, one dose is administered to said animal, preferably to said human at once, preferably by a single injection.

The invention provides a composition for use in a method of treating, ameliorating or preventing diabetes, preferably type II diabetes, said composition comprising: (a) a virus-like particle of an RNA bacteriophage with at least one first attachment site; and (b) at least one antigen with at least one second attachment site, wherein said at least one antigen comprises or preferably consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue; and wherein (a) and (b) are linked through said at least one first and said at least one second attachment site.

Preferably, said IL-1β mutein is linked to said virus-like particle of an RNA bacteriophage, so as to form an ordered and repetitive antigen-VLP array. In preferred embodiments of the invention, at least 20, preferably at least 30, more preferably at least 60, again more preferably at least 120 and further more preferably at least 180 IL-1β mutein molecules are linked to said virus-like particle of an RNA bacteriophage. In one preferred embodiment said virus-like particle of an RNA bacteriophage is a recombinant virus-like particle.

In one preferred embodiment the virus-like particle of an RNA bacteriophage comprises, consists essentially of, or alternatively consists of, recombinant coat proteins of an RNA bacteriophage. Preferably, the RNA bacteriophage is selected from the group consisting of: (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (1) bacteriophage PP7; (m) bacteriophage PRR1, and (n) bacteriophage AP205.

In a further preferred embodiment the virus-like particle of an RNA bacteriophage comprises, or alternatively consists essentially of, or alternatively consists of recombinant coat proteins of RNA bacteriophage Qβ. Virus-like particles of RNA bacteriophages, in particular of RNA bacteriophage Qβ, are disclosed in WO 02/056905. In particular, Example 18 of WO 02/056905 provides a detailed description of the preparation of VLPs of RNA bacteriophage Qβ.

In a further preferred embodiment the virus-like particle of an RNA bacteriophage comprises, or alternatively consists essentially of, or alternatively consists of recombinant coat proteins, wherein said recombinant coat proteins comprise or preferably consists of the amino acid sequence of SEQ ID NO:3.

In one preferred embodiment, the compositions and vaccines of the invention have an antigen density being from 0.5 to 4.0. The term “antigen density”, as used herein, refers to the average number of IL-1β mutein molecules which is linked per subunit, preferably per coat protein, of the VLP of an RNA bacteriophage, preferably of RNA-bacteriophage Qβ. Thus, this value is calculated as an average over all the subunits of the VLP of an RNA bacteriophage, in the composition or vaccines of the invention.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. Preferably, the at least one first attachment site is a lysine residue, pointing to or being on the exterior of the VLP. In a further preferred embodiment said first attachment site is an amino group of a lysine residue of SEQ ID NO: 3. In a further preferred embodiment said first attachment site is the amino group of any one of the lysine residues in positions 2, 13, 16, 46, 60, 63, and 67 of SEQ ID NO:3. In a further preferred embodiment said first attachment site is the amino group of any one of the lysine residues of the coat protein, preferably of SEQ ID NO:3, which are exposed to the exterior of the capsid.

In a further embodiment said IL-1β mutein consists of a mutated amino acid sequence, wherein the amino acid sequence to be mutated is human IL-1β, and wherein preferably the amino acid sequence to be mutated is SEQ ID NO:4, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by a lysine residue. Thus, in a very preferred embodiment said IL-1β mutein consists of the amino acid sequence of SEQ ID NO:6.

Typically and preferably said at least one antigen with at least one second attachment site is produced by way of recombinant expression, preferably by way of expression in a bacterial system, most preferably in E. coli. Said at least one antigen with at least one second attachment site may comprise an amino acid linker wherein said amino acid linker comprises said second attachment site. Additionally or alternatively, said at least one antigen with at least one second attachment site may comprise a tag, such as His tag, Myc tag, Fc tag or HA tag in order to facilitate purification.

In a preferred embodiment said virus-like particle of an RNA bacteriophage with at least one first attachment site and said at least one antigen with at least one second attachment site are linked by way of a covalent bond, preferably by way of a non-peptide covalent bond. In a further preferred embodiment said first attachment site and said second attachment site are linked by way of a covalent bond, preferably by way of a non-peptide covalent bond.

In a further preferred embodiment only one of said second attachment sites associates with said first attachment site through at least one non-peptide covalent bond leading to a single and uniform type of binding of said antigen to said virus-like particle of an RNA bacteriophage, wherein said only one second attachment site that associates with said first attachment site is a sulfhydryl group, and wherein said antigen and said virus-like particle of an RNA bacteriophage interact through said association to form an ordered and repetitive antigen array, and wherein further preferably said first attachment site is an amino group of a lysine residue.

In a preferred embodiment the first attachment site comprises, or preferably is, an amino group, preferably the amino group of a lysine residue. In another preferred embodiment the second attachment site comprises, or preferably is, a sulfhydryl group, preferably a sulfhydryl group of a cysteine residue.

In a further preferred embodiment, said first attachment is an amino group and said second attachment site is a sulfhydryl group. In a still further preferred embodiment, said first attachment is an amino group of a lysine residue, and said second attachment site is a sulfhydryl group of a cysteine residue.

In a preferred embodiment said at least one antigen with at least one second attachment site is linked to the VLP by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the VLP, and a further functional group which can react with the preferred second attachment site, preferably with a sulfhydryl group, most preferably with a sulfhydryl group of cysteine residue(s) inherent of, or artificially added to the IL-1β mutein, and optionally also made available for reaction by reduction. The heterobifunctional cross-linker is preferably selected from the group consisting of SMPH (Pierce), Sulfo-MBS, Sulfo-EMCS, Sulfo-GMBS, Sulfo-SIAB, Sulfo-SMPB, Sulfo-SMCC, SVSB, and SIA. The above mentioned cross-linkers all lead to formation of an amide bond after reaction with the amino group and a thioether linkage with the sulfhydryl groups. Most preferably, said hetero-bifunctional cross-linker is succinimidyl-6-[β-maleimidopropionamido]hexanoate (SMPH).

In a further preferred embodiment said at least one antigen with said at least one second attachment site further comprises a linker, wherein said linker comprises said second attachment site, and wherein said linker is associated with said antigen by way of a peptide bond. In a preferred embodiment said linker is associated to the IL-1β mutein by way of at least one covalent bond, preferably by at least one, preferably one peptide bond. Preferably, the linker comprises, or alternatively consists of, the second attachment site. In a further preferred embodiment, the linker comprises a sulfhydryl group, preferably of a cysteine residue. In another preferred embodiment, the amino acid linker is a cysteine residue.

In a further preferred embodiment the linker is added to the C-terminus of the IL-1β mutein. Preferred linkers according to this invention are glycine linkers (G)n further containing a cysteine residue as second attachment site. In a very preferred embodiment said linker is GGCG (SEQ ID NO:7) or GGC (SEQ ID NO:8), preferably GGCG (SEQ ID NO:7). In a still further preferred embodiment said linker is GGCG (SEQ ID NO:7), wherein said linker is added to the C-terminus of said IL-1β mutein.

In a further preferred embodiment said linker further comprises a His-tag, wherein preferably said His-tag is positioned between said IL-1β mutein and the C-terminal cystein-containing glycine linker. Thus, in a very preferred embodiment said linker comprises or preferably consists of LEHHHHHHGGCG (SEQ ID NO:9) or LEHHHHHHGGC (SEQ ID NO:10), wherein most preferably said linker consists of LEHHHHHHGGCG (SEQ ID NO:9). In a further preferred embodiment said linker consists of LEHHHHHHGGCG (SEQ ID NO:9), wherein said linker is added to the C-terminus of the IL-1β mutein.

In a preferred embodiment, said at least one antigen with at least one second attachment site consists of any one of SEQ ID NOs 11 to 14 or 21. In a very preferred embodiment, said at least one antigen with at least one second attachment site consists of any one of SEQ ID NOs 11 to 14, wherein preferably said at least one antigen with at least one second attachment site consists of any one of SEQ ID NOs 11 or 12. Most preferably, said antigen with at least one second attachment site consists of SEQ ID NO:11.

One or several antigen molecules, i.e. IL-1 molecules, can be attached to one subunit of the VLP, preferably of RNA bacteriophage coat proteins, preferably through the exposed lysine residues of the coat proteins of RNA bacteriophage VLP, if sterically allowable. A specific feature of the VLPs of RNA bacteriophage and in particular of the Qβ coat protein VLP is thus the possibility to couple several antigens per subunit. This allows for the generation of a dense antigen array.

It was found that the stability of the inventive compositions can be improved by the addition of a salt. Thus, in a preferred embodiment the composition of the invention further comprising a stabilizer, wherein preferably said stabilizer is an inorganic salt. In a further preferred embodiment said stabilizer is sodium chloride or potassium chloride, most preferably sodium chloride. In a further preferred embodiment the concentration of said stabilizer, preferably of said inorganic salt, and most preferably of sodium chloride in said composition is 5 to 200 mM, more preferably 10 to 100 mM, and still more preferably 25 to 75 mM, and most preferably 50 mM. In a very preferred embodiment the concentration of sodium chloride in said composition is 5 to 200 mM, more preferably the concentration of sodium chloride in said composition is 10 to 100 mM, still more preferably the concentration of sodium chloride in said composition is 25 to 75 mM, and most preferably the concentration of sodium chloride in said composition is 50 mM.

It was also found that the solubility of the compositions of the invention in aqueous solutions can be improved by the addition of a non-ionic surfactant, preferably of polysorbat 20 or polysorbat 80. Thus, in a further preferred embodiment the composition of the invention further comprises a non-ionic surfactant, wherein preferably said non-ionic surfactant is polysorbat 20 or polysorbat 80, and wherein further preferably said non-ionic surfactant is polysorbat 20. In a further preferred embodiment the composition of the invention comprises said non-ionic surfactant in a concentration of 0.01 to 0.5 mg/ml, preferably 0.05 to 0.25 mg/ml, and further preferably 0.10 mg/ml. In a very preferred embodiment the composition of the invention comprises a non-ionic surfactant, wherein said non-ionic surfactant is polysorbat 20, and wherein the concentration of polysorbat 20 in said pharmaceutical composition is 0.01 to 0.5 mg/ml, and wherein further preferably the concentration of polysorbat 20 in said pharmaceutical composition is 0.05 to 0.25 mg/ml, and wherein still further preferably the concentration of polysorbat 20 in said pharmaceutical composition is 0.10 mg/ml.

In one aspect, the invention provides a vaccine composition comprising any one of the compositions of the invention. In a further aspect, the invention provides a vaccine composition for the treatment of type II diabetes, said vaccine composition comprising or alternatively consisting of an effective amount of a composition comprising: (a) a virus-like particle of an RNA bacteriophage with at least one first attachment site; and (b) at least one antigen with at least one second attachment site, wherein said at least one antigen consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue; and wherein (a) and (b) are linked through said at least one first and said at least one second attachment site.

An effective amount of a composition of the invention is an amount which is capable of inducing an immune response, preferably an immune response against human IL-1β, in the treated subject, preferably in a human, and wherein said immune response results in a therapeutic or prophylactic effect in diabetes, preferably in type II diabetes.

In one embodiment, the vaccine composition further comprises at least one adjuvant, preferably aluminum hydroxide. However, an advantageous feature of the present invention is the high immunogenicity of the composition, even in the absence of adjuvant. Therefore, in a preferred embodiment, the vaccine composition is devoid of adjuvant. The absence of an adjuvant, furthermore, minimizes the occurrence of unwanted inflammatory T-cell responses representing a safety concern in the vaccination against self antigens. Thus, the administration of the vaccine composition of the invention to a patient will preferably occur without administering at least one adjuvant to the same patient prior to, simultaneously or after the administration of the vaccine composition. However, when an adjuvant is administered, the administration of the at least one adjuvant may hereby occur prior to, simultaneously or after the administration of the inventive composition or of the vaccine composition.

When the composition and/or the vaccine composition of the invention is administered to an individual, it may be in a form which contains salts, buffers, adjuvants, or other substances which are desirable for improving the efficacy of the conjugate. Examples of materials suitable for use in preparation of vaccines or pharmaceutical compositions are provided in numerous sources including Remington's Pharmaceutical Sciences (Osol, A, ed., Mack Publishing Co., (1990)). This includes sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

The vaccines of the invention are said to be “pharmaceutically acceptable” if their administration can be tolerated by a recipient individual, preferably by a human. Further, the vaccines of the invention are administered in a “therapeutically effective amount” (i.e., an amount that produces a desired physiological effect). The nature or type of immune response is not a limiting factor of this disclosure. Without the intention to limit the present invention by the following mechanistic explanation, the inventive vaccine composition might induce antibodies which bind to IL-1β and thus reduce its concentration and/or interfering with its physiological or pathological function.

In a further aspect, the invention provides a pharmaceutical composition comprising: any one of the composition or vaccine compositions of the invention; and (b) a pharmaceutically acceptable carrier.

In a further aspect, the invention provides a pharmaceutical composition for use in a method of treating diabetes, preferably type II diabetes, said pharmaceutical composition comprising: (a) a virus-like particle of an RNA bacteriophage with at least one first attachment site; (b) at least one antigen with at least one second attachment site, wherein said at least one antigen consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue; and wherein (a) and (b) are linked through said at least one first and said at least one second attachment site; and (c) a pharmaceutically acceptable carrier.

Thus, the invention provides a method for the treatment, amelioration and/or prevention of diabetes, preferably of type II diabetes, said method comprising administering any one of the compositions, vaccine compositions, or pharmaceutical compositions of the invention to an animal, preferably to a human. In particular, the invention provides a method for the treatment, amelioration and/or prevention of diabetes, preferably of type II diabetes, said method comprising administering to an animal, preferably to a human, a composition comprising: (a) a virus-like particle of an RNA bacteriophage with at least one first attachment site; and (b) at least one antigen with at least one second attachment site, wherein said at least one antigen consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue; and wherein (a) and (b) are linked through said at least one first and said at least one second attachment site.

The invention further provides a method for the treatment, amelioration and/or prevention of diabetes, preferably of type II diabetes, said method comprising administering to an animal, preferably to a human, a pharmaceutical composition comprising: (a) a virus-like particle of an RNA bacteriophage with at least one first attachment site; and (b) at least one antigen with at least one second attachment site, wherein said at least one antigen consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue; and wherein (a) and (b) are linked through said at least one first and said at least one second attachment site.

In a further preferred embodiment said method comprises administering to an animal, preferably to a human, a pharmaceutical composition, wherein a single dose of said pharmaceutical composition comprises 1 to 1500 μg of total protein, wherein preferably said total protein consists or is composed of (a) said virus-like particle of an RNA bacteriophage with at least one first attachment site; and (b) said at least one antigen with at least one second attachment site. In a further preferred embodiment a single dose of said medicament comprises 5 to 1000 μg, more preferably 5 to 900 μg, still more preferably 5 to 600 μg, still more preferably 5 to 400 μg, still more preferably 10 to 300 μg, still more preferably 10 to 100 μg of total protein. In a further preferred embodiment a single dose of said medicament comprises 10, 30, 100 or 300 μg of total protein, wherein most preferably said single dose comprises 100 μg of total protein, and wherein said total protein consists of (a) said virus-like particle of an RNA bacteriophage with at least one first attachment site; and (b) said at least one antigen with at least one second attachment site. In a further preferred embodiment a single dose of said medicament further comprises 0.1 to 5 mg, preferably 0.2 to 2 mg, more preferably 0.5 to 1.5 mg, and most preferably 1.0 mg of aluminum hydroxide.

With respect to the methods of the invention, said composition, said vaccine and/or said pharmaceutical composition is administered to said animal, preferably to said human, in an immunologically effective amount.

In one embodiment, the compositions, vaccine compositions and/or pharmaceutical compositions are administered to said animal, preferably to said human, by injection, infusion, inhalation, oral administration, or other suitable physical methods. In a preferred embodiment, the compositions, vaccine compositions and/or pharmaceutical compositions are administered to said animal, preferably to said human, intramuscularly, intravenously, transmucosally, transdermally, intranasally, intraperitoneally, subcutaneously, or directly into the lymphe node.

A further aspect of the invention is the use of the compositions, the vaccine compositions and/or of the pharmaceutical compositions described herein for the treatment, amelioration and/or prevention of diabetes, preferably of type II diabetes.

A further aspect of the invention is the use of the compositions, the vaccine compositions and/or of the pharmaceutical compositions described herein for the manufacture of a medicament for the treatment, amelioration and/or prevention of diabetes, preferably of type II diabetes. In more detail, the invention provides for the use of a composition for the manufacture of a medicament for the treatment, amelioration and/or prevention of diabetes, preferably of type II diabetes, said composition comprising: (a) a virus-like particle of an RNA bacteriophage with at least one first attachment site; and (b) at least one antigen with at least one second attachment site, wherein said at least one antigen consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue; and wherein (a) and (b) are linked through said at least one first and said at least one second attachment site.

In a further preferred embodiment a single dose of said medicament comprises 1 to 1500 μg of total protein, wherein preferably said total protein consists or is composed of (a) said virus-like particle of an RNA bacteriophage with at least one first attachment site; and (b) said at least one antigen with at least one second attachment site. In a further preferred embodiment a single dose of said medicament comprises 5 to 1000 μg, more preferably 5 to 900 μg, still more preferably 5 to 600 μg, still more preferably 5 to 400 μg, still more preferably 10 to 300 μg, still more preferably 10 to 100 μg of total protein. In a further preferred embodiment a single dose of said medicament comprises 10, 30, 100 or 300 μg of total protein, wherein most preferably said single dose comprises 100 μg of total protein, and wherein said total protein consists of (a) said virus-like particle of an RNA bacteriophage with at least one first attachment site; and (b) said at least one antigen with at least one second attachment site. In a further preferred embodiment a single dose of said medicament further comprises 0.1 to 5 mg, preferably 0.2 to 2 mg, more preferably 0.5 to 1.5 mg, and most preferably 1.0 mg of aluminum hydroxide.

It is to be understood that all technical features and embodiments described herein, in particular those described for the compositions of the invention, may be applied to all aspects of the invention, especially to the vaccine compositions, pharmaceutical compositions, methods and uses, alone or in any possible combination. In this context it is explicitly underlined that the following embodiments of said at least one antigen with at least one second attachment site are particularly preferred:

In a very preferred embodiment said composition, vaccine composition, or pharmaceutical composition comprises: (a) a virus-like particle of an RNA bacteriophage with at least one first attachment site, wherein said RNA bacteriophage is bacteriophage Qβ; and (b) at least one antigen with at least one second attachment site, wherein said at least one antigen consists of an IL-1β mutein, wherein said IL-1β mutein consists of SEQ ID NO:6; and wherein (a) and (b) are linked through said at least one first and said at least one second attachment site.

In a very preferred embodiment said composition, vaccine composition, or pharmaceutical composition comprises: (a) a virus-like particle of an RNA bacteriophage with at least one first attachment site, wherein said virus-like particle of an RNA bacteriophage comprises, essentially consists of, or alternatively consists of SEQ IN NO:3; and (b) at least one antigen with at least one second attachment site, wherein said at least one antigen consists of an IL-1β mutein, wherein said IL-1β mutein consists of SEQ ID NO:6; and wherein (a) and (b) are linked through said at least one first and said at least one second attachment site.

In a very preferred embodiment said composition, vaccine composition, or pharmaceutical composition comprises: (a) a virus-like particle of an RNA bacteriophage with at least one first attachment site, wherein said RNA bacteriophage is bacteriophage Qβ; and (b) at least one antigen with at least one second attachment site, wherein said at least one antigen with at least one second attachment site is SEQ ID NO:11; and wherein (a) and (b) are linked through said at least one first and said at least one second attachment site.

In a very preferred embodiment said composition, vaccine composition, or pharmaceutical composition comprises: (a) a virus-like particle of an RNA bacteriophage with at least one first attachment site, wherein said virus-like particle of an RNA bacteriophage comprises, essentially consists of, or alternatively consists of SEQ IN NO:3; and (b) at least one antigen with at least one second attachment site, wherein said at least one antigen with at least one second attachment site is SEQ ID NO:11; and wherein (a) and (b) are linked through said at least one first and said at least one second attachment site.

In a very preferred embodiment said composition, vaccine composition, or pharmaceutical composition comprises: (a) a virus-like particle of an RNA bacteriophage with at least one first attachment site, wherein said RNA bacteriophage is bacteriophage Qβ; and (b) at least one antigen with at least one second attachment site, wherein said at least one antigen consists of an IL-1β mutein, wherein said IL-1β mutein consists of SEQ ID NO:6; and wherein (a) and (b) are linked through said at least one first and said at least one second attachment site, and wherein said first attachment site is an amino group of a lysine residue, and said second attachment site is a sulfhydryl group of a cysteine residue, and wherein said first attachment site is linked to said second attachment site via at least one non-peptide covalent bond.

In a very preferred embodiment said composition, vaccine composition, or pharmaceutical composition comprises: (a) a virus-like particle of an RNA bacteriophage with at least one first attachment site, wherein said virus-like particle of an RNA bacteriophage comprises, essentially consists of, or alternatively consists of SEQ IN NO:3; and (b) at least one antigen with at least one second attachment site, wherein said at least one antigen consists of an IL-1β mutein, wherein said IL-1β mutein consists of SEQ ID NO:6; and wherein (a) and (b) are linked through said at least one first and said at least one second attachment site, and wherein said first attachment site is an amino group of a lysine residue, and said second attachment site is a sulfhydryl group of a cysteine residue, and wherein said first attachment site is linked to said second attachment site via at least one non-peptide covalent bond.

In a very preferred embodiment said composition, vaccine composition, or pharmaceutical composition comprises: (a) a virus-like particle of an RNA bacteriophage with at least one first attachment site, wherein said RNA bacteriophage is bacteriophage Qβ; and (b) at least one antigen with at least one second attachment site, wherein said at least one antigen with at least one second attachment site is SEQ ID NO:11; and wherein (a) and (b) are linked through said at least one first and said at least one second attachment site, and wherein said first attachment site is an amino group of a lysine residue, and said second attachment site is a sulfhydryl group of a cysteine residue, and wherein said first attachment site is linked to said second attachment site via at least one non-peptide covalent bond.

In a very preferred embodiment said composition, vaccine composition, or pharmaceutical composition comprises: (a) a virus-like particle of an RNA bacteriophage with at least one first attachment site, wherein said virus-like particle of an RNA bacteriophage comprises, essentially consists of, or alternatively consists of SEQ IN NO:3; and (b) at least one antigen with at least one second attachment site, wherein said at least one antigen with at least one second attachment site is SEQ ID NO:11; and wherein (a) and (b) are linked through said at least one first and said at least one second attachment site, and wherein said first attachment site is an amino group of a lysine residue, and said second attachment site is a sulfhydryl group of a cysteine residue, and wherein said first attachment site is linked to said second attachment site via at least one non-peptide covalent bond.

In a very preferred embodiment said composition, vaccine composition, or pharmaceutical composition further comprises a stabilizer, wherein said stabilizer is an inorganic salt, preferably sodium chloride, and wherein preferably the concentration of said stabilizer in said composition, vaccine composition, or pharmaceutical composition is 5 to 200 mM, more preferably 10 to 100 mM, and still more preferably 25 to 75 mM, and wherein most preferably 50 mM.

In a very preferred embodiment said composition, vaccine composition, or pharmaceutical composition further comprises a stabilizer, wherein said stabilizer is sodium chloride, and wherein preferably the concentration of sodium chloride in said composition, vaccine composition, or pharmaceutical composition is 5 to 200 mM, more preferably 10 to 100 mM, and still more preferably 25 to 75 mM, and wherein most preferably 50 mM.

In a very preferred embodiment said composition, vaccine composition, or pharmaceutical composition further comprises a non-ionic surfactant, wherein preferably said non-ionic surfactant is polysorbat 20, and wherein further preferably the concentration of said non-ionic surfactant in said composition, vaccine composition, or pharmaceutical composition is 0.01 to 0.5 mg/ml, preferably 0.05 to 0.25 mg/ml, and still further preferably 0.10 mg/ml.

In a very preferred embodiment said composition, vaccine composition, or pharmaceutical composition further comprises a non-ionic surfactant, wherein said non-ionic surfactant is polysorbat 20, and wherein the concentration of said polysorbat 20 in said composition, vaccine composition, or pharmaceutical composition is 0.01 to 0.5 mg/ml, preferably 0.05 to 0.25 mg/ml, and still further preferably 0.10 mg/ml.

In a very preferred embodiment said composition, vaccine composition, or pharmaceutical composition comprises: (a) a virus-like particle of an RNA bacteriophage with at least one first attachment site, wherein said virus-like particle of an RNA bacteriophage comprises, essentially consists of, or alternatively consists of SEQ IN NO:3; and (b) at least one antigen with at least one second attachment site, wherein said at least one antigen with at least one second attachment site is SEQ ID NO:11; and wherein (a) and (b) are linked through said at least one first and said at least one second attachment site, and wherein said first attachment site is an amino group of a lysine residue, and said second attachment site is a sulfhydryl group of a cysteine residue, and wherein said first attachment site is linked to said second attachment site via at least one non-peptide covalent bond, and wherein said composition, vaccine composition, or pharmaceutical composition further comprises a stabilizer, wherein said stabilizer is an inorganic salt, preferably sodium chloride, and wherein further preferably the concentration of sodium chloride in said composition, vaccine composition, or pharmaceutical composition is 5 to 200 mM, more preferably 10 to 100 mM, and still more preferably 25 to 75 mM, and wherein most preferably 50 mM.

In a very preferred embodiment said composition, vaccine composition, or pharmaceutical composition comprises: (a) a virus-like particle of an RNA bacteriophage with at least one first attachment site, wherein said virus-like particle of an RNA bacteriophage comprises, essentially consists of, or alternatively consists of SEQ IN NO:3; and (b) at least one antigen with at least one second attachment site, wherein said at least one antigen with at least one second attachment site is SEQ ID NO:11; and wherein (a) and (b) are linked through said at least one first and said at least one second attachment site, and wherein said first attachment site is an amino group of a lysine residue, and said second attachment site is a sulfhydryl group of a cysteine residue, and wherein said first attachment site is linked to said second attachment site via at least one non-peptide covalent bond, and wherein said composition, vaccine composition, or pharmaceutical composition further comprises a non-ionic surfactant, wherein preferably said non-ionic surfactant is polysorbat 20, and wherein further preferably the concentration of polysorbat 20 in said composition, vaccine composition, or pharmaceutical composition is 0.01 to 0.5 mg/ml, preferably 0.05 to 0.25 mg/ml, and still further preferably 0.10 mg/ml.

In a further preferred embodiment said compositions, said vaccine compositions, and or said pharmaceutical compositions are administered to an animal, preferably to a human, wherein a single dose administered to said animal, preferably to said human, comprises of 1 to 1500 μg of total protein, wherein preferably said total protein consists or is composed of (a) said virus-like particle of an RNA bacteriophage with at least one first attachment site; and (b) said at least one antigen with at least one second attachment site. In a further preferred embodiment said single dose comprises 5 to 1000 μg, more preferably 5 to 900 μg, still more preferably 5 to 600 μg, still more preferably 5 to 400 μg, still more preferably 10 to 300 μg, still more preferably 10 to 100 μg of total protein. In a further preferred embodiment said single dose comprises 10, 30, 100 or 300 μg of total protein, wherein most preferably said single dose comprises 100 μg of total protein, and wherein said total protein consists of (a) said virus-like particle of an RNA bacteriophage with at least one first attachment site; and (b) said at least one antigen with at least one second attachment site. In a further preferred embodiment said single dose further comprises 0.1 to 5 mg, preferably 0.2 to 2 mg, more preferably 0.5 to 1.5 mg, and most preferably 1.0 mg of aluminum hydroxide.

### Example 1

### Cloning, Expression and Purification of hIL-1β₁₁₆₋₂₆₉ and hIL-1β₁₁₆₋₂₆₉ (D145K)

Human IL-1β₁₁₆₋₂₆₉ and the IL-1β mutein hIL-1β₁₁₆₋₂₆₉ (D145K) were cloned, expressed and purified following the procedure disclosed in Example 10A and 10B of WO2008/037504A1.

### Example 2

### A. Biological Activity of hIL-1β₁₁₆₋₂₆₉ and hIL-1β₁₁₆₋₂₆₉ (D145K) in Mice

Three female C3H/HeJ mice per group were injected intravenously with 10 μg of either the wild type human IL-1β₁₁₉₋₂₆₉ protein or hIL-1β₁₁₆₋₂₆₉ (D145K) mutein. Serum samples were withdrawn before and 3 h after injection and analyzed for the relative increase in the concentration of the pro-inflammatory cytokine IL-6. Mice injected with the wild type human IL-1β₁₁₉₋₂₆₉ protein showed an increase of 2.38±0.69 ng/ml in serum IL-6 concentrations, whereas mice injected with hIL-1β₁₁₆₋₂₆₉ (D145K) mutein showed an increase of 1.39±0.26 ng/ml in serum IL-6 concentrations.

### B. Biological Activity of hIL-1β₁₁₆₋₂₆₉ and hIL-1β₁₁₆₋₂₆₉ (D145K) in Human PBMC

Peripheral blood mononuclear cells (PBMCs) were isolated from heparinized blood of a healthy donor by Ficoll density gradient centrifugation. 5×10⁵ cells per well were incubated with titrating amounts of either hIL-1β₁₁₆₋₂₆₉ or hIL-1β116-269 (D145K). The results are shown in Table 1.

TABLE 1
Biological activity of human IL-1β₁₁₆₋₂₆₉ and
IL-1β₁₁₆₋₂₆₉ (D145K) mutein in human PBMC.

| | Protein/mutein | |
|---|---|---|
| | concentration | |
| | (in ng/ml) required to | |
| | induce 600 pg/ml | Fold reduction in |
| | IL-6 from human | bioactivity relative to |
| Protein/mutein | PBMC | wild type hIL-1β₁₁₆₋₂₆₉ |
| hIL-1β₁₁₆₋₂₆₉ | 2 | —/— |
| hIL-1β₁₁₆₋₂₆₉ (D145K) | 386 | 169 |

### Example 3

### A. Coupling of Human IL-1β₁₁₆₋₂₆₉ and Human IL-1β₁₁₆₋₂₆₉ (D145K) Mutein to Qβ Virus-Like Particles

Chemical cross-linking of the wild type human IL-1β₁₁₉₋₂₆₉ protein and the human IL-1β₁₁₆₋₂₆₉ (D145K) was performed essentially following the procedure disclosed in Example 2A of WO2008/037504A1.

### B. Immunization of Mice with Human IL-1β₁₁₆₋₂₆₉ and IL-1β₁₁₆₋₂₆₉ (D145K) Mutein Coupled to Qβ Capsid

Four female balb/c mice per group were immunized with Qβ coupled to either the wild type hIL-1β₁₁₆₋₂₆₉ protein or one of the IL-1β₁₁₆₋₂₆₉ (D145K) mutein. Fifty μg of total protein were diluted in PBS to 200 μl and injected subcutaneously (100 μl on two ventral sides) on day 0, 14 and 28. Mice were bled retroorbitally on day 35, and sera were analyzed using ELISAs specific for either for IL-1β₁₁₆₋₂₆₉ (D145K) mutein or the wild type human IL-1β₁₁₆₋₂₆₉ protein.

### C ELISA

ELISA plates were coated either with the wild type hIL-1β₁₁₆₋₂₆₉ protein or IL-β₁₁₆₋₂₆₉ (D145K) mutein, respectively, at a concentration of 1 μg/ml. The plates were blocked and then incubated with serially diluted mouse sera from day 35. Bound antibodies were detected with enzymatically labeled anti-mouse IgG antibody. Antibody titers of mouse sera were calculated as the average of those dilutions which led to half maximal optical density at 450 nm, and are shown in Table 2.

TABLE 2
Anti-hIL-1β₁₁₆₋₂₆₉ (wild type and mutein)-specific IgG titers raised by
immunization with Qβ-hIL-1β₁₁₆₋₂₆₉ or Qβ-hIL-1β₁₁₆₋₂₆₉ mutein vaccines.

| | Average | Average |
|---|---|---|
| | anti-hIL-1β₁₁₆₋₂₆₉ | anti-hIL-1β₁₁₆₋₂₆₉ |
| | wild type | mutein |
| Vaccine | IgG titer (±SD) | IgG titer (±SD) |
| Qβ-hIL-1β₁₁₆₋₂₆₉ | 253325 ± 184813 | —/— |
| Qβ-hIL-1β₁₁₆₋₂₆₉ (D145K) | 78365 ± 26983 | 93241 ± 28856 |

Qβ-hIL-1β₁₁₆₋₂₆₉-immunization induced high titers of IgG antibodies against hIL-1β₁₁₆₋₂₆₉. Moreover, vaccination with Qβ-hIL-1β₁₁₆₋₂₆₉ mutein vaccine induced high IgG titers against both the Qβ-hIL-1β₁₁₆₋₂₆₉ mutein used as immunogen, and the wild type hIL-1β₁₁₆₋₂₆₉ protein.

### D. In Vitro Neutralization of Human IL-1β

Sera of mice immunized with Qβ coupled to either wild type hIL-1β₁₁₆₋₂₆₉ protein or to Qβ-hIL-1β₁₁₆₋₂₆₉ mutein were tested for their ability to inhibit the binding of human IL-1β protein to its receptor. ELISA plates were therefore coated with a recombinant human IL-1receptorI-hFc fusion protein at a concentration of 1 μg/ml, and co-incubated with serial dilutions of the above mentioned sera and 100 ng/ml of hIL-1β₁₁₆₋₂₆₉ protein. Binding of hIL-1β₁₁₆₋₂₆₉ to the immobilized human IL-1receptorI-hFc fusion protein was detected with a biotinylated anti-human IL-1β antibody and horse radish peroxidase conjugated streptavidin. All sera raised against Qβ-hIL-1β₁₁₆₋₂₆₉ mutein vaccine completely inhibited the binding of 100 ng/ml wild type hIL-1β₁₁₆₋₂₆₉ to hIL-1RI at serum concentrations ≧3.3%.

The same sera were also tested for their ability to inhibit the hIL-1β₁₁₆₋₂₆₉-induced secretion of IL-6 from human cells. Human PBMCs were therefore prepared as described in EXAMPLE 2B and incubated with 10 ng/ml wild type hIL-1β₁₁₆₋₂₆₉, which had been premixed with titrating concentrations of the sera described above. After over night incubation the cell culture supernatants were analyzed for the presence of IL-6. The neutralizing capacity of the sera was expressed as those dilutions which lead to half maximal inhibition of IL-6 secretion. In order to allow a direct comparison to the neutralizing capacity of the serum raised against wild type hIL-1β₁₁₆₋₂₆₉, the neutralizing titers of the sera raised against Qβ-hIL-1β₁₁₆₋₂₆₉ mutein were corrected for the respective ELISA titers measured against wild type hIL-1β₁₁₆₋₂₆₉ (see Table 2). As shown in Table 3 the sera raised against hIL-1β₁₁₆₋₂₆₉ mutein were able to inhibit the secretion of IL-6 induced by wild type hIL-1β₁₁₆₋₂₆₉.

TABLE 3
Neutralizing titer determined in sera of mice immunized with
IL-1 beta muteins.

| | Neutralizing titer (corrected for ELISA titer |
|---|---|
| Vaccine | against wild type hIL-1β₁₁₆₋₂₆₉) |
| Qβ-hIL-1β₁₁₆₋₂₆₉ | 3333 |
| Qβ-hIL-1β₁₁₆₋₂₆₉ (D145K) | 2369 |

### E. In Vivo Neutralization of IL-1β

The in vivo neutralizing capacity of the antibodies raised by immunization with Qβ coupled to either wild type hIL-1β₁₁₆₋₂₆₉ protein or to one of the hIL-1β₁₁₆₋₂₆₉ D145K mutein is investigated. Three female C3H/HeJ mice per group are therefore immunized three times on days 0, 14, and 28 with 50 μg of either vaccine. On day 35 all immunized mice are injected intravenously with 1 μg of free wild type hIL-1β₁₁₆₋₂₆₉. As a control three naive mice are injected at the same time with the same amount of wild type hIL-1β₁₁₆₋₂₆₉. As readout of the inflammatory activity of the injected hIL-1β₁₁₆₋₂₆₉, serum samples are withdrawn immediately before and 3 h after injection and analyzed for the relative increase in the concentration of the pro-inflammatory cytokine IL-6. Whereas naive mice show a strong increase in serum IL-6 concentrations 3 h after injection of hIL-1β₁₁₆₋₂₆₉, all mice immunized with Qβ coupled to the wild type hIL-1β₁₁₆₋₂₆₉ protein or to one of the hIL-1β₁₁₆₋₂₆₉ mutein do not show any increase in serum IL-6, indicating that the injected hIL-1β₁₁₆₋₂₆₉ is efficiently neutralized by the antibodies induced by the vaccines.

### Example 4

### Amelioration of Diet-Induced Type II Diabetes in Male C57BL/6 Mice (Prophylactic Setting)

Mouse IL-1α₁₁₅₋₂₇₀ was cloned following the procedure disclosed in Example 15 of WO2008/037504A1 and coupled to Qβ VLP. Mouse IL-1β₁₁₉₋₂₆₉ was cloned following the procedure disclosed in Example 1 of WO2008/037504A1 and coupled to Qβ VLP. Male C57BL/6 mice were immunized on days 0, 14, and 28 with 50 μg of either Qβ, Qβ-mIL-1α₁₁₅₋₂₇₀, Qβ-mIL-1β₁₁₉₋₂₆₉ or a mixture of 50 μg Qβ-mIL-1α₁₁₅₋₂₇₀ and 50 μg Qβ-mIL-1β (n=16 per group). All mice were fed normal rodent chow (Provimi Kliba no. 3436: 18.5% protein, 4.5% fat, 4.5% fiber, 6.5% ash, 54% carbohydrates) during the immunization period. On day 35 this diet was replaced by a high fat diet (Provimi Kliba no. 2127: 23.9% protein, 35% fat, 4.9% fiber, 5% ash, 23.2% carbohydrates) for half of the mice of each group (n=8) while the other half (n=8) was kept on normal chow. Five months after the last immunization mice fed the high-fat diet were obese (average body weight>45 g) and showed elevated fasting glucose levels (Table 4, 0′).

In order to investigate on the diabetic phenotype of these mice an oral glucose tolerance test was performed by administering a dose of 2 mg/g body weight of D-glucose intragastrically and determining blood glucose levels at regular intervals using the Accu-check blood glucose meter (Roche). Table 4 shows that Qβ-immunized mice on normal chow showed an initial peak of 291.5 mg/dl in blood glucose levels after 15 minutes which was followed by a sharp drop and a complete return to pre-challenge levels within 90 minutes. The peak levels and kinetics of this response were essentially identical in all mouse groups that had been maintained on normal chow (Table 4). Qβ-immunized mice on high fat diet on the other hand peaked at higher levels (367.9 mg/dl) and failed to show a significant decline until 60 min post-challenge; only thereafter blood glucose levels started to decrease, without however returning to baseline levels within the 2 hour observation period. This severe impairment in glucose clearance indicates that obese Qβ-immunized mice had developed a diabetic phenotype. Obese Qβ-mIL-1α-, Qβ-mIL-1β-, or double immunized mice showed an initial increase in blood glucose levels to ˜350 mg/dl, which was immediately followed by a sustained decline, resulting in glucose levels that were consistently lower than in obese Qβ-immunized control mice. When calculating the area under the curves resulting form the repeated glucose measurements shown in table 4, it becomes evident, that obese Qβ-mIL-1α-, Qβ-mIL-1β-, or double immunized mice manifested an improved glucose clearance with respect to obese Qβ-immunized control mice (Table 5). Taken together these data show that immunization with Qβ-mIL-1α or Qβ-mIL-1β or a combination of both resulted in a clear amelioration of the diet-induced diabetic phenotype.

TABLE 4
Blood glucose levels (mg/dl; mean ± SEM) before and at different time points after
intragastric administration of 2 mg/g glucose (Mice were fasted for a period of 5 hours before
the experiment).

| Mouse group | 0′ | 15′ | 30′ | 45′ | 60′ | 90′ | 120′ |
|---|---|---|---|---|---|---|---|
| Qβ | 194.3 ± 8.0 | 342.4 ± 20.9 | 367.9 ± 28.3 | 356.9 ± 29.3 | 350.4 ± 31.6 | 298.5 ± 31.9 | 250.3 ± 23.3 |
| high fat diet | | | | | | | |
| Qβ-mIL-1α₁₁₅₋₂₇₀ | 195.6 ± 4.5 | 356.6 ± 7.4 | 324.3 ± 21.4 | 320.6 ± 26.9 | 303.3 ± 26.6 | 234.9 ± 19.6 | 208.6 ± 13.2 |
| high fat diet | | | | | | | |
| Qβ-mIL-1β₁₁₉₋₂₆₉ | 200.1 ± 11.6 | 341.9 ± 11.6 | 348.3 ± 27.6 | 313.3 ± 31.2 | 297.6 ± 27.3 | 256.9 ± 22.6 | 234.7 ± 19.0 |
| high fat diet | | | | | | | |
| Qβ-mIL-1α₁₁₅₋₂₇₀/Qβ-mIL-1β₁₁₉₋₂₆₉ | 190.6 ± 10.4 | 337.9 ± 27.1 | 309.9 ± 44.2 | 284.3 ± 47.6 | 281.6 ± 45.8 | 240.6 ± 40.8 | 221.0 ± 37.3 |
| high fat diet | | | | | | | |
| Qβ | 154.4 ± 3.5 | 291.5 ± 14.7 | 210.0 ± 10.0 | 193.6 ± 7.2 | 183.1 ± 6.2 | 149.5 ± 4.7 | 134.0 ± 4.2 |
| normal chow | | | | | | | |
| Qβ-mIL-1α₁₁₅₋₂₇₀ | 171.6 ± 4.8 | 297.4 ± 12.6 | 230.4 ± 10.9 | 203.0 ± 11.4 | 192.6 ± 11.7 | 158.0 ± 8.5 | 136.4 ± 6.2 |
| normal chow | | | | | | | |
| Qβ-mIL-1β₁₁₉₋₂₆₉ | 158.0 ± 3.6 | 312.3 ± 6.8 | 229.0 ± 10.8 | 188.3 ± 4.4 | 172.6 ± 4.9 | 145.8 ± 7.6 | 134.0 ± 3.8 |
| normal chow | | | | | | | |
| Qβ-mIL-1α₁₁₅₋₂₇₀/Qβ-mIL-1β₁₁₉₋₂₆₉ | 152.4 ± 4.4 | 283.6 ± 8.4 | 220.3 ± 11.3 | 188.5 ± 11.1 | 187.8 ± 9.0 | 149.1 ± 8.6 | 131.5 ± 6.9 |
| normal chow | | | | | | | |

TABLE 5
Glucose clearance in immunized mice. The area under the curve (AUC)
resulting from the consecutive glucose measurements represented in
Table 10 was calculated for each individual mouse.
Group means of AUC are expressed with SEM.

| Mouse group | Normal chow | High fat diet |
|---|---|---|
| Qβ | 4120 ± 460 | 14746 ± 2262 |
| Qβ-mIL-1α₁₁₅₋₂₇₀ | 6104 ± 2313 | 10184 ± 1800 |
| Qβ-mIL-1β₁₁₉₋₂₆₉ | 4276 ± 419 | 10459 ± 1699 |
| Qβ-mIL-1α₁₁₅₋₂₇₀/Qβ-mIL-1β₁₁₉₋₂₆₉ | 4464 ± 531 | 9500 ± 3382 |

### Example 5

### Amelioration of Diet-Induced Type II Diabetes in Male C57BL/6 Mice

The DNA sequence encoding amino acids 119-269 of mouse IL-1β was amplified by PCR from cDNA of TNFα-activated murine macrophages using oligonucleotides IL1BETA-3 (5′-ATATATGATATCCCCATTAGACAGCTGCACTACAGG-3; SEQ ID NO:15) and IL1BETA-2 (5′-ATATATCTCGAGGGAAGACACAGATTCCATGGTGAAG-3′; SEQ ID NO:16) and cloned into the vector pET42T (EXAMPLE 10 of WO2008/037504A1). The resulting plasmid pET42T-mIL-1β119-269 encodes the mature mouse IL-1β protein fused to a hexahistidine tag and a cysteine containing linker at the C-terminus. Due to the introduction of the EcoRV restriction site the valine residue at the N-terminus of mouse IL-1β is substituted by a short N-terminal extension consisting of three amino acids (MDI). By site directed mutagenesis of the plasmid pET42T-mIL-1β116-269, an expression vector was constructed which encodes the murine version of the human IL-1β mutein hIL-1β116-269 (D145K) (SEQ ID NO:6) with the C-terminal tag of SEQ ID NO:10, namely mIL-1β116-269 (D145K) (SEQ ID NO:17). The mutation was introduced using the Quik-Change® Site directed mutagenesis kit (Stratagene) and the oligonucleotides D143K-1 (5′-CAGTGGTCAG GACATAATTA AATTCACCAT GGAATCTGTGTC-3′; SEQ-ID:18) and D143K-2 (5′-GACACAGATT CCATGGTGAA TTTAATTATG TCCTGACCACTG-3′; SEQ ID NO:19). Expression and purification of the mutein mIL-1β116-269 (D145K) was performed following the procedures disclosed in WO2008/037504A1.

Groups of male C57BL/6 mice (8 weeks of age, n=8) were immunized subcutaneously on days 0, 14, 28, 42, and 147 with 50 μg of either Qβ or Qβ-mIL-1β119-269(D145K). Starting with day 0, one half of the mice (n=16) was put on a high fat diet (Provimi Kliba no. 2127: 23.9% protein, 35% fat, 4.9% fiber, 5% ash, 23.2% carbohydrates), while the other half (n=16) was maintained on normal diet (Provimi Kliba no. 3436: 18.5% protein, 4.5% fat, 4.5% fiber, 6.5% ash, 54% carbohydrates) throughout the experiment. After eight months mice fed the high-fat diet were obese (average body weight>45 g) and showed elevated fasting glucose levels (Table 6).

In order to investigate on the diabetic phenotype of the mice on high fat diet an oral glucose tolerance test was performed by administering a dose of 2 mg/g body weight of D-glucose intragastrically and determining blood glucose levels at regular intervals using the Accu-check blood glucose meter (Roche). Table 7 shows that Qβ-immunized mice on high fat diet peaked at 318.5 mg/dl 30 minutes after injection and failed to show a significant decline until 60 min post-challenge; only thereafter blood glucose levels started to decrease, without however returning to baseline levels within the 2 hour observation period. This severe impairment in glucose clearance indicates that obese Qβ-immunized mice had developed a diabetic phenotype. Obese Qβ-mIL-1β119-269(D145K)-immunized mice showed an initial increase in blood glucose levels to 318.6 mg/dl, which was immediately followed by a sustained decline, resulting in glucose levels that were consistently lower than in obese Qβ-immunized control mice. Two hours after challenge blood glucose levels had returned to pre-challenge levels in these mice. When calculating the area under the curves resulting form the repeated glucose measurements shown in Table 7, it becomes evident, that obese Qβ-mIL-1β119-269 (D145K)-immunized mice manifested an improved glucose clearance with respect to obese Qβ-immunized control mice (Table 8). Taken together these data show that immunization with Qβ-mIL-1β119-269 (D145K) resulted in a clear amelioration of the diet-induced diabetic phenotype.

TABLE 6
Average body weights and fasting blood glucose levels
after 5 hours fasting (means ± SEM).

| | Average | Fasting blood |
|---|---|---|
| | body weight | glucose levels |
| | (g) | (mg/dl) |
| Qβ high fat diet | 47.16 ± 2.24 | 185.9 ± 6.3 |
| Qβ-mIL-1β₁₁₉₋₂₆₉(D145K) high fat diet | 51.08 ± 1.23 | 194.0 ± 4.2 |
| Qβ normal chow | 36.95 ± 0.97 | 148.4 ± 6.5 |
| Qβ-mIL-1β₁₁₉₋₂₆₉(D145K) normal chow | 36.23 ± 1.30 | 147.0 ± 3.1 |

TABLE 7
Blood glucose levels (mg/dl; mean ± SEM) before and at different time
points after intragastric administration of 2 mg/g glucose. Mice were fasted
for a period of 5 hours before the experiment.

| Mouse group | 0′ | 15′ | 30′ | 45′ | 60′ | 90′ | 120′ |
|---|---|---|---|---|---|---|---|
| Qβ | 185.9 ± 6.3 | 315.1 ± 15.5 | 318.5 ± 23.1 | 305.5 ± 24.4 | 316.8 ± 33.5 | 238.4 ± 21.8 | 220.1 ± 15.3 |
| high fat diet | | | | | | | |
| Qβ-mIL-1β₁₁₉₋₂₆₉(D145K) | 194.0 ± 4.2 | 318.6 ± 18.3 | 290.0 ± 15.7 | 278.6 ± 12.5 | 280.1 ± 10.4 | 218.4 ± 7.8 | 199.4 ± 7.5 |
| high fat diet | | | | | | | |

TABLE 8
Glucose clearance in immunized mice. The area under the curve
(AUC) resulting from the consecutive glucose measurements
represented in Table 7 was calculated for each individual mouse.
Group means of AUC are expressed with SEM.
Peaks below baseline were excluded form the analysis.

| Mouse group | AUC |
|---|---|
| Qβ high fat | 11060 ± 1895 |
| Qβ-mIL-1β₁₁₉₋₂₆₉(D145K) high fat | 7375 ± 539 |

### Example 6

### Influence of Primary Structure Variations in hIL-1β Mutein Constructs on Biological Function

The human IL-1β mutein construct of SEQ ID NO:11 differs from human IL-1β (SEQ ID NO:6) in the following structural elements: (a) replacement of the N-terminal alanine residue by the N-terminal extension sequence MDI, (b) the mutation D145K, and a linker sequence at the C-terminus including (c) the hexahistidine-tag LEHHHHHH, and (d) the cysteine containing sequence GGCG. In order to assess the influence of each of these sequence elements on the functional properties of the molecule, different variants of the construct were generated and compared by means of biological activity and in-vitro receptor binding.

Seven different constructs were generated as indicated in Table 9. Construct “MDI-D145K-His6” corresponds to SEQ ID NO:11. MA-wt corresponds to the interleukin-1β wild type sequence containing an N-terminal methionine (SEQ ID NO:20). Presence of the N-terminal extension is indicated by “MDI” instead of “MA”, presence of the “D145K mutation is indicated by “D145K” instead of “wt”, and presence of the C-terminal linker sequence LEHHHHHHGGCG by His6. For construct “MDI-D145K-CG” and construct “MA-wt-CG” the C-terminal linker sequence is replaced by the sequence CG.

Biological activities of all protein variants were determined in two independent cell-based in vitro assays. The mutation D145K is known to reduce the biological activity of IL-113 wild type without affecting the affinity to IL1-receptor type I. Because biological activity is expected to correlate to at least some degree to the reactogenicity of IL-1β in vivo, this mutation is also expected to reduce potential toxic effects when used as a pharmaceutical. Both activity assays, namely IL-1β-induced IL-6 release from HeLa cells and a cytotoxicity assay using human A375 melanoma cells show corresponding results. The introduction of mutation D145K and the N-terminal extension MDI both have strong reducing effects on the bioactivity of the constructs. The combination of both sequence modifications results in even less active protein variants with an approximately 10⁵-fold reduced bioactivity. The introduction of the hexahistidine tag does not result in significant effects on the activity of the protein. Importantly, none of the introduced modifications cause an increase of protein bioactivity.

Furthermore, differences in receptor binding affinity of all protein variants were determined by a homogeneous time-resolved fluorescence assay (HTRF), clearly indicating that the activity reduction caused by the N-terminal extension MDI is related to a reduced binding affinity to the IL-1 receptor. Modifications on the C-terminus have no impact on affinity and only marginal effects are observed for the mutation D145K. The determined reduction factors for bioactivity and the IC₅₀ values determined in receptor binding studies are summarized in Table 9.

TABLE 9
Bioactivity measurements and in-vitro receptor binding of constructs comprising
different combinations of structural elements. For the IL-6 release assay, bioactivity reduction
is expressed as the ratio of the determined EC50 value of the respective protein variant and
the determined LC50 value of the wild type protein (MA-wt) derived from a four-parameter
logistic fit. Correspondingly, the bioactivity reduction factors determined by the A375
cytotoxicity assay are defined as the ratio of the LC50 value determined for the respective
protein variant and the EC50 value of the wild type protein (MAwt).

| | | | | MA- | | MDI- | MDI- |
|---|---|---|---|---|---|---|---|
| | | MAwt- | MA-wt- | D145K- | MDI- | D145K- | D145K- |
| construct | MA-wt | CG | His6 | His6 | wt-His6 | CG | His6 |
| Bioactivity reduction (IL6 | 1 | n.d. | 3 | 2 × 10² | 43 | 6 × 10⁴ | 6 × 10⁴ |
| release, HeLa cells) | | | | | | | |
| Bioactivity reduction | 1 | 27 | 25 | 4 × 10³ | 7 × 10² | 6 × 10⁵ | 6 × 10⁵ |
| (Cytotoxicity, A375 cells) | | | | | | | |
| IC50 [nM] Receptor binding | 20 | 31 | 23 | 28 | 1340 | 6769 | 4812 |
| (HTRF) | | | | | | | |

### Example 7

### Reactogenicity of MDI-D145K-His6 (SEQ ID NO:11) in Rhesus Monkeys

An in vivo study was performed in primates to assess the reactogenicity of the mutein IL-1β relative to wt IL-1β. Naïve, Rhesus monkeys (Macaca mulatta) were intravenously administered either human wild-type IL-1β, rhesus monkey IL-1β or human MDI-D145K-His6 (SEQ ID NO:11) over a range of concentrations. IL-6 concentrations were measured in sera as readout for IL-1β activity. Over several days, groups of rhesus monkeys (1 ♂ and 1 ♀ per group) were i.v. administered 0.1, 0.3, 1.0 and 1.5 μg/kg of either human IL-1β or rhesus IL-1β or 0.3, 1.0, 10 and 100 μg/kg of MDI-D145K-His6. Sera were drawn for cytokine analysis 3 hrs after administration of IL-1β of MDI-D145K-His6. The bioactivity of MDI-D145K-His6 was reduced approximately 7.5-fold relative to wild type IL-1β.

### Example 8

### Impact of Salt Concentration on Vaccine Stability

In order to assess the influence of NaCl on the stability of the vaccine during processing, vaccine batches comprising Qβ VLP coupled with antigen were produced containing various concentrations of NaCl (0 mM, 25 mM, 50 mM, 75 mM). Integrity of the vaccine was assessed by SE-HPLC (analysis on a Dionex HPLC system with a TSKgel 5000PWXL column). A concentration-dependent influence of NaCl on the particle integrity was observed (Table 10). A concentration of 50 mM NaCl was required to achieve less than 1% degradation.

TABLE 10
Influence of NaCl concentration on the stability during processing
of the vaccine as determined by SE-HPLC. Results of the
batch 1 are means of two independent batches (batch 1a:
97.2% rel area main peak, 2.8% rel. area degradation,
batch 1b: 97.4% rel area main peak, 2.6% rel. area degradation).

| | NaCl | Main peak | Degradation |
|---|---|---|---|
| Batch | [mM] | [% rel. area] | [% rel. area] |
| 1 | 0 | 97.3 | 2.7 |
| 2 | 25 | 99.0 | 1.0 |
| 3 | 50 | 99.3 | 0.7 |
| 4 | 75 | 99.4 | 0.6 |

### Example 9

### Impact of Surfactant Concentration on Vaccine Solubility

Vaccine preparations comprising 1.9 mg/ml Qβ VLP coupled with antigen (human IL-1β mutein, SEQ ID NO:11) were generated with different concentrations of Polysorbat 20 and exposed to shearing forces by intensive pipetting. In the presence of 0.05 mg/ml Polysorbat 20 intensive pipetting resulted in the formation of visible filament like particles in the vaccine preparation. In the presence of 0.10 mg/ml Polysorbat 20 the preparation remained a clear solution after intensive pipetting. The formation of visible filament like particles was not observed when 0.10 mg/ml Polysorbat 20 was present in the preparation.

### Example 10

### Reactogenicity of Qβ-MDI-D145K-His6 and Qβ-rhesusMDI-D145K-His6 in Rhesus Monkeys

Three groups of Rhesus monkeys (n=12, 6 male and 6 female) received 6 biweekly subcutaneous injections of 300 μg of either Qβ alone, Qβ coupled to MDI-D145K-His6 (SEQ ID NO:11) or a rhesus monkey specific version thereof, Qβ coupled to rhesusMDI-D145K-His6 (SEQ ID NO:21), each in combination with Alhydrogel as adjuvant (1.0 mg/dose Al(OH)₃). As readout for the reactogenicity of the vaccines, IL-6 concentrations were determined in serum three hours after the first and third vaccine injection, respectively. Low levels of IL-6, barely above the detection limit of the assay (20 pg/ml), were measured in 1/12, 5/12 and 4/12 animals after the first injection of Qβ, Qβ-rhesusMDI-D145K-His6 or Qβ-MDI-D145K-His6, respectively. Two animals receiving Qβ-MDI-D145K-His6 had slightly higher levels. There was no IL-6 response of note after the third immunisation. In contrast, large increases in serum concentrations of IL-6 (˜2000 pg/ml) were recorded in Qβ-immunized control animals three hours after intravenous administration of 1 μg/kg wt IL-1β.

### Example 11

### Immunogenicity of Qβ-MDI-D145K-His6 and Qβ-rhesusMDI-D145K-His6 in Rhesus Monkeys

IgG ELISA titers specific for human wt IL-1β were measured at different time points in sera of rhesus monkeys that had been immunized with Qβ-MDI-D145K-His6 or Qβ-rhesusMDI-D145K-His6 as described in EXAMPLE 10. Table 11 shows that high titers of human IL-1β-specific IgG antibody titers were induced by both vaccines, which peaked after the 5^{th} injection and then declined approximately 5 to 7 fold in the following 6 weeks.

TABLE 11
Anti-human IL-1β specific IgG ELISA titers (GMT ± SEM) raised by Qβ-MDI-
D145K-His6 and Qβ-rhesusMDI-D145K-His6 in Rhesus monkeys. Titers are expressed as
the reciprocal of those serum dilutions which lead to half-maximal OD at 450 nm in ELISA.

| | Day | | | | | | | | |
|---|---|---|---|---|---|---|---|---|---|
| | 1 | 15 | 29 | 43 | 57 | 71 | | | |
| | Vaccine injection | | | | | | | | |
| | X | X | X | X | X | X | 85 | 108 | 114 |
| Qβ-MDI- | n.d. | n.d. | n.d. | 15982 ± 3008 | 19025 ± 2171 | 28762 ± 5424 | 24099 ± 3987 | 5616 ± 1169 | 4124 ± 898 |
| D145K-His6 | | | | | | | | | |
| Qβ- | n.d. | n.d. | n.d. | 13915 ± 7212 | 16185 ± 8177 | 18674 ± 8147 | 18277 ± 10886 | 5772 ± 6565 | 3969 ± 5215 |
| rhesusMDI- | | | | | | | | | |
| D145K-His6 | | | | | | | | | |

n.d. = not determined

Sera from immunized monkeys were also tested for their ability to neutralize the biological activity of human wt IL-1β in vitro. HeLa cells were incubated with a constant amount of 6 pM wt IL-1β and serial dilutions of immune sera from different time points. IL-6 was measured in the cell culture supernatant as a readout of the biological activity of human wt IL-1β. Neutralizing activity could first be detected in sera after the third injection of vaccine and increased over time until reaching a peak after the sixth injection. In the following 4 weeks neutralizing titers then decreased approximately 2 to 3 fold (Table 12).

TABLE 12
Neutralizing titers (GMT ± SEM) induced by Qβ-MDI-D145K-His6 and Qβ-
rhesusMDI-D145K-His6 in rhesus monkeys. Titers were expressed as the reciprocal of those
serum dilutions that lead to half-maximal inhibition of the IL-1β-induced IL-6 release. The
lower limit of quantification (LLOQ) was 35.

| | Day | | | | | | | | |
|---|---|---|---|---|---|---|---|---|---|
| | 1 | 15 | 29 | 43 | 57 | 71 | | | |
| | Vaccine injection | | | | | | | | |
| | X | X | X | X | X | X | 85 | 108 | 114 |
| Qβ-MDI- | <LLOQ | <LLOQ | <LLOQ | 62 ± 30 | 407 ± 184 | 1310 ± 578 | 2377 ± 2057 | 780 ± 562 | 684 ± 368 |
| D145K- | | | | | | | | | |
| His6 | | | | | | | | | |
| Qβ- | <LLOQ | <LLOQ | <LLOQ | 82 ± 74 | 314 ± 522 | 740 ± 2817 | 1127 ± 4157 | 667 ± 3327 | 570 ± 2131 |
| rhesusMDI- | | | | | | | | | |
| D145K- | | | | | | | | | |
| His6 | | | | | | | | | |

In order to determine the neutralizing activity of the antibodies induced by Qβ-MDI-D145K-His6 or Qβ-rhesusMDI-D145K-His6 in vivo, two weeks after the sixth vaccine injection half the animals of each group and of the Qβ-immunized control group was challenged by an intravenous injection of 1 μg/kg wt IL-1β. IL-6 concentrations in serum were determined 3, 6, and 9 hours after challenge as readout of the biological activity of wt IL-1β. As shown in Table 13 Qβ-immunized monkeys mounted a strong IL-6 response which peaked at 3 hours and then declined to near background levels by 9 hours after injection. In contrast, for animals immunised with Qβ-MDI-D145K-His6 and Qβ-rhesusMDI-D145K-His6 IL-6 remained below the limit of detection at all time points after IL-1β injection, showing that the antibodies induced by these vaccines had neutralized the biological activity of IL-1β.

TABLE 13
Neutralization of biological activity of IL-1β in vivo by antibodies
induced by Qβ-MDI-D145K-His6 and Qβ-rhesusMDI-D145K-His6
in rhesus monkeys. Serum IL-6 was quantified by a multiplex cytokine
bead array kit. Values are expressed in pg/ml serum. The
lower limit of quantification (LLOQ) was 20 pg/ml.

| | hours after challenge | | | |
|---|---|---|---|---|
| | 0 | 3 | 6 | 9 |
| Qβ | <LLOQ | 2091 ± 436 | 675 ± 174 | 96 ± 20 |
| Qβ-MDI-D145K-His6 | <LLOQ | <LLOQ | <LLOQ | <LLOQ |
| Qβ-rhesusMDI-D145K- | <LLOQ | <LLOQ | <LLOQ | <LLOQ |
| His6 | | | | |

1. A method of treating type II diabetes, said method comprising administering a composition to an animal, wherein said composition comprises:
(a) a virus-like particle of an RNA bacteriophage with at least one first attachment site; and
(b) at least one antigen with at least one second attachment site, wherein said at least one antigen consists of an IL-1β mutein, wherein said IL-1β mutein consists of a mutated amino acid sequence, wherein the amino acid sequence to be mutated is human IL-1β, and wherein the N-terminal amino acid residue of said amino acid sequence to be mutated is replaced by the amino acid sequence MDI (SEQ ID NO:5), and wherein the amino acid residue in position 145 of said amino acid sequence to be mutated is exchanged by another amino acid residue;
and wherein (a) and (b) are linked through said at least one first and said at least one second attachment site.

2. The method of claim 1, wherein said amino acid sequence to be mutated is SEQ ID NO:4.

3. The method of claim 1, wherein the amino acid residue in position 145 of said amino acid sequence to be mutated is exchanged by a lysine residue.

4. The method of claim 1, wherein said IL-β mutein consists of the amino acid sequence of SEQ ID NO:6.

5. The method of claim 1, wherein said at least one antigen with at least one second attachment site comprises:
(i) said IL-1β mutein; and
(ii) a linker, wherein said linker comprises said second attachment site, and wherein said linker comprises GGCG (SEQ ID NO:7).

6. The method of claim 1, wherein said at least one antigen with at least one second attachment site is SEQ ID NO 11, SEQ ID NO:12, SEQ ID NO:13 or SEQ ID NO:14.

7. The method of claim 1,
wherein said RNA bacteriophage is bacteriophage Qβ.

8. The method of claim 1, wherein said virus-like particle of an RNA bacteriophage comprises recombinant coat proteins of an RNA bacteriophage, wherein said recombinant coat proteins consist of SEQ ID NO:3.

9. The method of claim 1, wherein said first attachment site is linked to said second attachment site via at least one covalent bond, wherein said at least one covalent bond is a non-peptide bond.

10. (canceled)

11. (canceled)

12. The method of claim 1, wherein said first attachment is an amino group of a lysine residue, and said second attachment site is a sulfhydryl group of a cysteine residue.

13. The method of claim 1, wherein only one of said second attachment sites associates with said first attachment site through at least one non-peptide covalent bond leading to a single and uniform type of binding of said antigen to said virus-like particle, wherein said only one second attachment site that associates with said first attachment site is a sulfhydryl group, and wherein said antigen and said virus-like particle interact through said association to form an ordered and repetitive antigen array.

14. The method of claim 1, wherein said least one first attachment site and said at least one second attachment site are covalently linked via a heterobifunctional cross-linker.

15. The method of claim 1, wherein said composition further comprises a stabilizer, wherein said stabilizer is an inorganic salt, and wherein the concentration of said stabilizer in said composition is 25 to 75 mM.

16. The method of claim 1, wherein said composition further comprises a non-ionic surfactant, wherein said non-ionic surfactant is polysorbat 20, and wherein the concentration of said non-ionic surfactant in said composition is 0.05 to 0.25 mg/ml.

17. A vaccine composition comprising an effective amount of the composition of claim 1.

18. (canceled)

19. (canceled)

20. The vaccine composition of claim 17, wherein said vaccine composition comprises an adjuvant.

21. A pharmaceutical composition comprising:
(a) the composition of claim 1; and
(b) a pharmaceutically acceptable carrier.

22. (canceled)

23. (canceled)

24. (canceled)

25. (canceled)

26. (canceled)

27. (canceled)

28. The composition of claim 1, wherein said composition is administered to an animal, wherein a single dose administered to said animal, comprises of 1 to 1500 μg of total protein, wherein said total protein consists of (a) said virus-like particle of an RNA bacteriophage with at least one first attachment site; and (b) said at least one antigen with at least one second attachment site.

29. The composition claim 28, wherein said single dose further comprises 0.1 to 5 mg of aluminum hydroxide.

30. The method of claim 1, wherein said at least one antigen with at least one second attachment site is SEQ NO: 11.


SEQUENCE LISTING (21 sequences)

SEQ ID NO: 1 - protein, 569 aa, Homo sapiens [sequence omitted]

SEQ ID NO: 2 - protein, 398 aa, Homo sapiens
  MISC_FEATURE (1)..(398): IL-1 receptor type II
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 3 - protein, 132 aa, bacteriophage Qbeta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 4 - protein, 153 aa, Homo sapiens
  MISC_FEATURE (1)..(153): IL-1beta 116-269
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 5 - protein, 3 aa, artificial sequence
  chemically synthesized N-terminal sequence
MDI

SEQ ID NO: 6 - protein, 155 aa, Homo sapiens
  MISC_FEATURE (1)..(155): IL-1beta 116-269 MDI D145K
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSS

SEQ ID NO: 7 - protein, 4 aa, artificial sequence
  chemically synthesized GGCG linker
GGCG

SEQ ID NO: 8 - protein, 3 aa, artificial sequence
  chemically synthisized GGC linker
GGC

SEQ ID NO: 9 - protein, 12 aa, artificial sequence
  chemically synthesized His-CCGC linker
LEHHHHHHGGCG

SEQ ID NO: 10 - protein, 11 aa, artificial sequence
  chemically synthesized His-GGC linker
LEHHHHHHGGC

SEQ ID NO: 11 - protein, 167 aa, Homo sapiens
  MISC_FEATURE (1)..(167): IL-1beta MDI D145K his6 GGCG
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSSLEHHHHHHGGCG

SEQ ID NO: 12 - protein, 166 aa, Homo sapiens
  MISC_FEATURE (1)..(166): IL-1beta MDI D145K his6 GGC
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSSLEHHHHHHGGC

SEQ ID NO: 13 - protein, 159 aa, Homo sapiens
  MISC_FEATURE (1)..(159): IL-1beta MDI D145K his6 GGCG
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSSGGCG

SEQ ID NO: 14 - protein, 158 aa, Homo sapiens
  MISC_FEATURE (1)..(158): IL-1beta MDI D145K his6 GGC
MDIPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITKFTMQFVSSGGC

SEQ ID NO: 15 - DNA, 36 bp, artificial sequence
  chemically synthesized primer IL-1beta-3
atatatgatatccccattagacagctgcactacagg

SEQ ID NO: 16 - DNA, 37 bp, artificial sequence
  chemically synthesized primer IL-1beta-2
atatatctcgagggaagacacagattccatggtgaag

SEQ ID NO: 17 - protein, 165 aa, Mus musculus
  MISC_FEATURE (1)..(165): mIL-1beta 116-269 D145K
MDIPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIKFTMESVSSLEHHHHHHGGC

SEQ ID NO: 18 - DNA, 42 bp, artificial sequence
  chemically synthesized primer D143K-1
cagtggtcaggacataattaaattcaccatggaatctgtgtc

SEQ ID NO: 19 - DNA, 42 bp, artificial sequence
  chemically synthesized primer D143K-1
gacacagattccatggtgaatttaattatgtcctgaccactg

SEQ ID NO: 20 - protein, 154 aa, Homo sapiens
  MISC_FEATURE (1)..(154): IL-1beta 116-269
MAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 21 - protein, 167 aa, Macaca mulatta
  MISC_FEATURE (1)..(167): IL-1beta MDI D145K GGCG
MDIPVRSLHCTLRDAQLKSLVMSGPYELKALHLQGQDLEQQVVFSMSFVQGEESNDKIPVALGLKAKNLYLSCVLKDDKPTLQLESVDPKNYPKKKMEKRFVFNKIEINNKLEFESAQFPNWYISTSQAENMPVFLGGTRGGQDITKFTMQFVSSLEHHHHHHGGCG